(19) United States
(12) Patent Application Publication
(10) Pub. No.: US 2026/0309698 A1
(43) Pub. Date: Oct. 8, 2026

(54) DEVICE FOR SAMPLING A GAS, AND GAS MEASURING DEVICE FOR THE QUALITATIVE OR QUANTITATIVE DETERMINATION OF A COMPONENT IN A BREATHING GAS SAMPLE

(71) Applicant: Dräger Safety AG & Co. KGaA, Lübeck (DE)

(72) Inventors: Luis Brunswick Franco, Lübeck (DE); Tobias Reier, Lübeck (DE); Jens Rekow, Lübeck (DE); Malte Baesler, Lübeck (DE)

(21) Appl. No.: 19/636,720

(22) Filed: Apr. 1, 2026

(30) Foreign Application Priority Data
Apr. 2, 2025 (DE) 102025112800.6

(51) Int. Cl. G01N 33/497; A61B 5/097; G01N 1/24

(52) U.S. Cl. CPC G01N 33/4972; A61B 5/097; G01N 1/24; G01N 2001/241

(57) ABSTRACT

The present disclosure relates to a device for sampling a gas, and a gas measuring device for the qualitative or quantitative determination of a component in a breathing gas sample, with a corresponding device. In particular, the present disclosure may be used in a gas measuring device for carrying out a breath alcohol measurement. A device for sampling a gas is provided, the device comprising: a sampling element which defines a receiving volume for receiving a sample of the gas, an intake element which provides a variable intake volume and which is in fluidic connection with the receiving volume, and an actuator which is configured to change the intake volume via a mechanism and thus bring about a suctioning sampling into the receiving volume. In this case, the mechanism is designed as a locking mechanism.

### CROSS-REFERENCE TO RELATED APPLICATIONS

This application claims the priority of German Patent Application No. 102025112800.6, filed on Apr. 2, 2025, and titled “DEVICE FOR SAMPLING A GAS, AND GAS MEASURING DEVICE FOR THE QUALITATIVE OR QUANTITATIVE DETERMINATION OF A COMPONENT IN A BREATHING GAS SAMPLE” which is hereby incorporated by reference in its entirety for all nonlimiting purposes.

### TECHNICAL FIELD

The present disclosure relates to a device for sampling a gas, and a gas measuring device for the qualitative or quantitative determination of a component in a breathing gas sample with a corresponding device.

In particular, the present disclosure may be used in the field of breath alcohol measurement. In order to perform a breath alcohol measurement, a specific sample volume or a plurality of specific sample volumes are required, which can come from the upper airways or lungs of a subject. By detecting alcohol in the upper airways (mouth alcohol), it is possible to determine whether alcohol was ingested shortly beforehand through liquids or food. By taking a sample of air from the lungs (blood alcohol), the blood alcohol concentration can be determined, since the air in the lungs comes from the alveoli, which are in contact with the blood and constantly exchange gases, so that the blood-breath ratio can be determined. The terms breath alcohol and blood alcohol are often used synonymously.

### BACKGROUND

Devices for sampling a gas, in particular from the field of breath alcohol measurement, are known.

DE 10 2022 114 344 A1 discloses, in this respect, an analysis device and a method for analyzing a gas for a given substance. In this case, a sensor measures an indicator for the quantity or concentration of the substance in a gas sample located in a measuring chamber. A movement of an actuating unit in a first direction increases the volume of an intake chamber unit. A movement of the actuating unit in a second direction reduces the volume of the intake chamber unit. In this case, a motor rotates a conversion component about an axis. The conversion component is mechanically coupled to the actuating unit. Each rotational position of the conversion component relative to the axis determines a position of the actuating unit and thus a volume of the intake chamber unit.

DE 39 04 994 A1 discloses a device for conveying a gaseous sample into a measuring chamber of a detection device for determining the constituent of a component of a gas, in particular the proportion of alcohol in the breathing air, using a piston-cylinder unit as a conveying element, which conveys the sample from a gas supply via an inlet into the measuring chamber which is brought into flow communication with a sensor, the measurement-sensitive surface of the sensor forming a wall part of the displacement chamber of the conveying unit designed as a measuring chamber, and a detour along the measurement-sensitive surface of the sensor being provided in the course of the measurement sample path between the inlet and the displacement chamber.

DE 20 2004 020 736 U1 discloses a conveying device for conveying a fluid volume into or through a measuring chamber of a device for analyzing the fluid volume, the conveying device containing an electromotively operated piston-cylinder unit which is connected to a fluid reservoir, the piston of which has magnetic, electrical and/or electromagnetic properties and can be excited for conveying the fluid volume via a coil or coil element arranged around the cylinder.

U.S. Pat. No. 3,622,278 A discloses a device for collecting and analyzing the contents of a test subject's breath, comprising an input device through which the test subject exhales; a first collection device for collecting a predetermined amount of breath from the test subject, this predetermined amount being an estimate of the volume of the “dead air” spaces of the test subject's respiratory tract; second collection devices for collecting a further amount of breath from the test subject; first switching devices arranged between said input devices and said first collection devices, and also between said input devices and said second collection devices, said first switching devices serving to control the flow of breath from the test subject either to said first or said second collection devices; a first valve device that ensures the release of breath from the first collection device; a second switching device arranged between the input device and the second collection device and also between the input device and an outlet valve, the second switching device serving to control the flow of breath from the test subject either to the second collecting device or to the outlet valve; a gas analysis device for receiving and analyzing the gas content of the breath from the second collecting device; and a second valve device for controlling the flow of breath between the second collection chamber and the second collection chamber.

US 2012/0031165A1 discloses a method for taking breath samples, comprising the following steps: sending a drive signal to a piston pump in order to draw a breath sample into a collection chamber, the pump having an inlet line connected to the collection chamber; monitoring the operating state of the pump by monitoring the state of a switch designed to change its state if the pump piston is retracted into its pump housing as a suitable response of the pump to its drive signal, and to return to its original state if the pump piston is no longer retracted; using the state of the switch to confirm proper pump operation when drawing the breath sample into the chamber; and, if proper pump operation is confirmed, accepting the breath sample for further processing and analysis.

Further devices for sampling a gas, in particular from the field of breath alcohol measurement, are known from DE 60 300 172 T2, DE 10 316 333 B3, DE 10 2006 018 970 B3, U.S. Pat. Nos. 4,297,871 A, 6,923,040 B2 and EP 0 302 681 A2.

Commercially available breath alcohol measuring devices with devices for sampling a gas are known as the Alcotest and Interlock product families from Dräger.

### SUMMARY

It is an object of the present disclosure to provide an alternative to known devices for sampling a gas, and corresponding gas measuring devices, in particular a device and a corresponding gas measuring device with simple construction and simple control.

These and other objects are achieved by the subject matter of the claims. The claims, the description and the figures provide advantageous embodiments of the present disclosure.

According to the present disclosure, a device for sampling a gas is provided, the device comprising: a sampling element which defines a receiving volume for receiving a sample of the gas, an intake element which provides a variable intake volume and which is fluidically connected to the receiving volume, and an actuator which is configured to change the intake volume via a mechanism and thus bring about a suctioning sampling into the receiving volume. In this case, the mechanism is designed as a locking mechanism.

By providing a locking mechanism, it is possible, in a simple manner, to interrupt a drive movement of the actuator, in particular for a predetermined period of time, by locking (e.g., blocking, or acting as a detent) and thus to provide an engagement of the locking mechanism. During the locking process, the mechanism provides a defined position and thus a defined state of the intake volume. Therefore, it is not necessary for the actuator to have a position control means during the locking process. In summary, the device according to the present disclosure is therefore particularly simple in its construction and control.

The receiving volume of the sampling element can be constant or variable. For example, the sampling element can be a rigid body with a cavity which defines or contains the receiving volume and which can be in fluidic connection with the environment (e.g. the breathing airflow of a test subject) and with the intake element via a number of openings, which may be optionally closable. For example, the rigid body can have an inlet opening to the environment and an outlet opening to the intake element. If necessary, the cavity can have a further opening to one or more gas sensors.

The intake element can be designed in substantially any way. In some examples, however, the intake element is designed as a bellows, which is in direct or indirect (e.g. by means of a membrane) fluidic connection with the receiving volume, for example via an outlet opening of the sampling element. Other designs of the intake element are possible, for example as a piston pump. By changing the intake volume of the intake element, the intake element is configured to bring about a suctioning sampling process.

In the context of the present disclosure, an actuator is understood to be a device for providing kinetic energy for driving the mechanism, for example by converting hydraulic, pneumatic or electrical energy. In some examples, the actuator is configured to provide a linear drive movement or an approximately linear drive movement, and to act on the intake element via the mechanism.

The actuator can be provided, for example, as an electric linear motor, a pneumatic cylinder, a magnetic linear cylinder, a lifting magnet (for example with one or two stops) or a piezo motor. The actuator can also be provided as a motor with a rotating or pivoting drive movement and may include an intermediate mechanism (gear stage) interposed between a drive shaft of the motor and the mechanism, for converting the rotary movement into a linear movement or an approximate linear movement (including a thrust movement). Such an intermediate mechanism can, for example, include a cam disc, a rotary movement of the cam disc being converted into a linear movement or approximate linear movement of a plunger coupled to the cam disc.

Within the scope of the present disclosure, a locking mechanism is understood to be a transmission in which at least one transmission element is temporarily locked in its mobility relative to another transmission element, which may be a frame of the locking mechanism or another transmission element. This locking can take place in a form-fitting and/or force-fitting manner. The locking mechanism can be designed in particular as a ratchet, clamping mechanism, spring mechanism, switching mechanism or escapement mechanism.

In some examples, a first volume of the intake volume can be provided by means of the locking mechanism for a predetermined first period of time, it being possible for a second volume of the intake volume to be provided by means of the locking mechanism for a predetermined second period of time.

In this way, at least two predetermined volumes of the intake volume can be provided for respective predetermined periods of time, by means of the locking mechanism. By appropriately designing the components of the device, the volumes can be adapted to the requirements of the sampling and the corresponding measurement to be carried out. By appropriately selecting the predetermined periods of time, these can be adapted to the corresponding measurement to be carried out.

For example, the first volume can be provided by a rest position of the mechanism, while the second volume can be provided by a locking position (i.e. a position of the mechanism that it has in the state locked by the locking mechanism).

In some examples, the locking mechanism is designed as a tensioning-and-latching mechanism.

Within the scope of the present disclosure, a tensioning-and-latching mechanism can be understood as a transmission or a mechanism with a device for storing mechanical energy, in which, by means of a first actuation starting from a rest position to a locking position, mechanical energy is stored in the device and locking in the locking position is brought about, and, by means of a second actuation, the stored mechanical energy being released again and thus a return of the transmission to the rest position being brought about. The tensioning-and-latching mechanism thus provides the first volume in a first position, namely in the rest position, and the second volume in a second position, namely in the locking position.

An advantage of providing the tensioning-and-latching mechanism is that, at least for changing the position from the locking position to the rest position, no actuation by the actuator is necessary. This advantageously simplifies the control of the device and also advantageously reduces the energy consumption of the actuator.

The device for storing mechanical energy may be, for example, a spring element such as a spring. The device can be provided as an integral element of the actuator or separately from it. In some examples, in this respect, the actuator is provided as a lifting magnet with an integrated spring element.

In some examples, the tensioning-and-latching mechanism is designed as a releasable tensioning-and-latching mechanism (e.g., a trigger-releasable tensioning-and-latching mechanism).

A releasable tensioning-and-latching mechanism is understood to be a tensioning-and-latching mechanism in which a mechanical engagement of a transmission element of the tensioning-and-latching mechanism takes place against a restoring force of the device for storing mechanical energy, in order to provide the locked state of the tensioning-and-latching mechanism (i.e. in the second position or locking position) by engagement. By actuating, for example, the locking element or a tensioning element, the locking can be released, so that the previously described return to the rest position (or to an intermediate position or to a further intermediate position) can be brought about.

This advantageously represents a particularly easy-to-implement embodiment of a locking mechanism.

In some examples, the tensioning-and-latching mechanism comprises a tensioning element which is movable by the actuator and which can be brought into engagement with a locking element that is fixed or movable relative to a frame of the tensioning-and-latching mechanism, in particular pivotably mounted relative to the frame.

In this way, the locking can advantageously be revoked by actuating the actuator itself, which simplifies the design and control of the device.

In some examples, the locking element, which is fixed relative to the frame, is designed as a groove cam.

This advantageously represents a particularly easy-to-implement embodiment of the device. Furthermore, in this way it is particularly easily possible to provide the volumes required for the measurement to be carried out, by appropriately designing the geometric shape of the groove cam, which is easy to implement in terms of manufacturing technology.

In some examples, a third volume of the intake volume can be provided by means of the locking mechanism, it furthermore optionally being possible for a fourth volume of the intake volume to be provided by means of the locking mechanism.

In this way, the possible movement sequence of the device can be even better adapted to the measurement to be carried out, in that the volume-time profile that can be provided by the device allows further intermediate stages with corresponding additional positions of the mechanism.

In particular, the present disclosure can be used to particular advantage in the field of breath alcohol measurement. In order to determine both the mouth alcohol concentration and the blood alcohol concentration during a subject's exhalation process, it is necessary that two samples can be introduced into the sampling element in quick succession, which is particularly easy to do in this way.

In some examples, the tensioning element is mounted on a shaft of the actuator and has an engagement projection which engages with the groove cam, the groove cam having: a first region for providing a rest position of the tensioning element, which corresponds to a first volume of the intake volume, a second region for providing a locking position of the locking mechanism, which corresponds to a second volume of the intake volume, optionally a third region for providing an intermediate position of the locking mechanism, which corresponds to an optional third volume of the intake volume, and optionally a fourth region for providing a further intermediate position of the locking mechanism, which corresponds to an optional fourth volume of the intake volume.

This represents a design of the locking mechanism that is particularly simple in terms of construction and at the same time particularly flexible in adapting to the measurement to be carried out.

The locking (latching) of the tensioning element in the groove cam, as the locking element, can be achieved, for example, by the groove cam having at least one region, e.g. the second region, in which the mobility of the engagement projection of the tensioning element against the restoring force of the device for storing mechanical energy can be locked, i.e. latched, for example by means of a form-fitting connection.

The tensioning element can be mounted on the shaft for example at least rotatably or pivotably, for example by means of a sliding bushing. Alternatively, a rigid connection including integral design of the tensioning element and shaft is possible.

Each of the aforementioned regions, i.e., each of the the first region, the second region, the third region and the fourth region, as well as any possible further region, can be designed to be stable, metastable or unstable with respect to its blocking effect. In this case, it is preferred that the rest position is designed to be stable or metastable, i.e. that the tensioning element experiences substantially no change in position in the groove cam when a driving force of the actuator is removed, due to the restoring effect of the device for storing mechanical energy and the shaping of the groove cam. It is further preferred that the locking position is designed to be stable or metastable, i.e. that the tensioning element does not experience any change in position in the groove cam when a driving force of the actuator is removed, due to the restoring effect of the device for storing mechanical energy and the shaping of the groove cam. It is further preferred that the intermediate position is designed to be unstable, i.e. that the tensioning element does not remain in the intermediate position when a driving force of the actuator is removed, due to the restoring effect of the device for storing mechanical energy and the shaping of the groove cam, but rather assumes the locking position as a stable or metastable position. It is further preferred that the further intermediate position is designed to be unstable, i.e. that the tensioning element or the engagement projection does not remain in the further intermediate position when a driving force of the actuator is removed, due to the restoring effect of the device for storing mechanical energy and the shaping of the groove cam, but rather assumes the rest position as a stable or metastable position.

According to the present disclosure, a gas measuring device for the qualitative or quantitative determination of a component in a breathing gas sample by means of a gas sensor is further provided, the gas measuring device comprising the device for sampling a breathing gas as the gas.

The gas measuring device provides corresponding advantages and effects.

All features and advantages disclosed herein in connection with the device are also deemed to be disclosed in connection with the gas measuring device, and vice versa.

In some examples, the sampling element is configured to receive the gas sensor or to provide a fluidic connection between the receiving volume and the gas sensor.

Particularly preferably, in one embodiment of the present disclosure, the gas measuring device is provided as a breath alcohol measuring device, with an actuator in the form of a lifting magnet, the locking mechanism being designed as a tensioning-and-latching mechanism and converting the drive movement of the actuator into a linear movement for actuating the intake element. This allows high accuracy and reproducibility in the determination of mouth and blood alcohol concentration. Furthermore, the complexity of the breath alcohol measuring device is reduced compared to the prior art by decreasing the number of mechanical and electronic components required for operation, resulting in a breath alcohol measuring device that is simple in design, compact, and inexpensive.

A particular advantage of the present disclosure is that the intake volumes that can be provided (i.e. the first volume, the second volume, and optionally the third and fourth volumes) are fixedly predetermined, so that the reproducibility of the sampling is high without requiring displacement control (position control) of the actuator, and that the actuator can be deactivated in the rest position and in the locking position without an undesired change in the position of the tensioning element occurring.

### BRIEF DESCRIPTION OF THE DRAWINGS

These and other features, advantages and advantageous embodiments of the present disclosure can be found in the following description of the drawings, in which:

FIG. 1a is a first schematic exemplary embodiment according to the present disclosure of a gas measuring device with a device for sampling a gas, in a state in which the intake element has a second volume,

FIG. 1b shows the first exemplary embodiment according to FIG. 1a, in a state in which the intake element has a first volume,

FIG. 1c is a schematic detail view of a tensioning element of the exemplary embodiment according to FIGS. 1a and 1b,

FIG. 2a is a detailed plan view of a locking element designed as a groove cam,

FIG. 2b is a sectional view of the groove cam according to FIG. 2a along section line A-A, and

FIG. 3 is a schematic graph of intake volume-time profiles according to the present disclosure.

### DETAILED DESCRIPTION

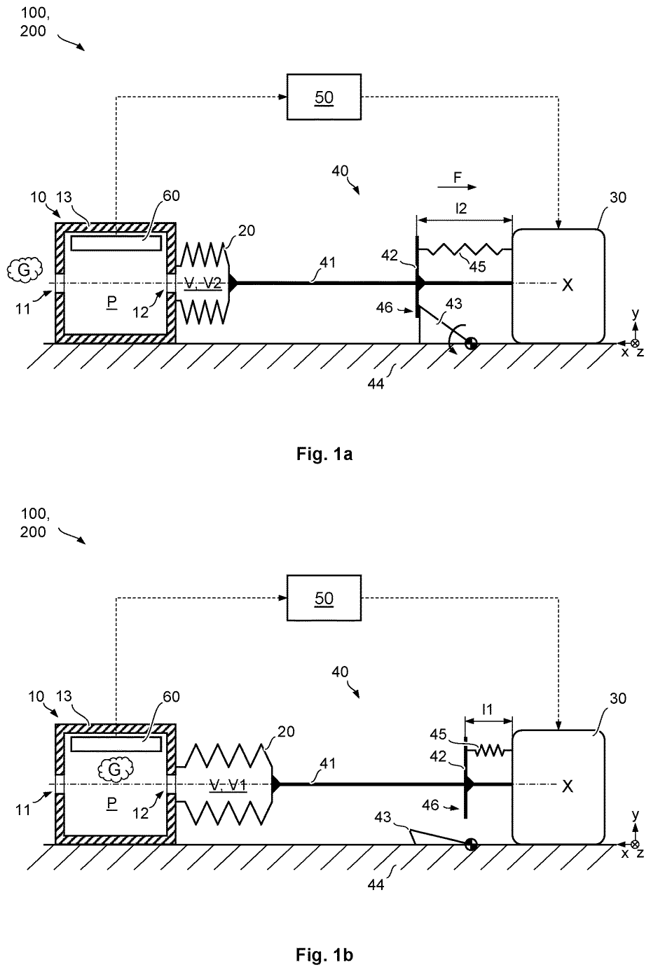

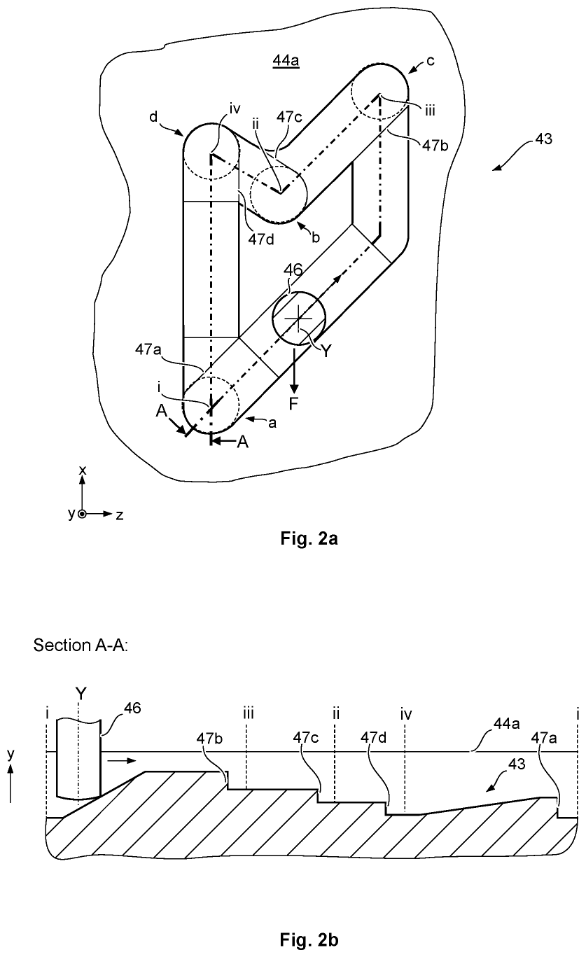

According to the present disclosure, a device 100 for sampling a gas G is provided. The device 100 can be represented as a component of a gas measuring device 200 for the qualitative or quantitative determination of a component in a breathing gas sample by means of a gas sensor 60, the device 100 in this case being configured for sampling a breathing gas as the gas G. A first exemplary embodiment of such a device 100 and such a gas measuring device 200 is shown in FIG. 1a and FIG. 1b. A detail of another exemplary embodiment of such a device 100 is shown in FIG. 2a, 2b. Unless otherwise stated, the following description refers to all possible embodiments of the present disclosure, both shown and not shown.

The device 100 according to the present disclosure comprises a sampling element 10, which defines a receiving volume P for receiving a sample of the gas G, an intake element 20, which provides a variable intake volume V and which is in fluidic connection with the receiving volume P, and an actuator 30, which is configured to change the intake volume V via a mechanism 40 and thus bring about a suctioning sampling into the receiving volume P. In this case, the mechanism 40 is designed as a locking mechanism 40. In a preferred embodiment, the locking mechanism 40 can be designed as a tensioning-and-latching mechanism 40, particularly preferably as a releasable tensioning-and-latching mechanism 40.

The sampling element 10 can, for example, comprise a housing 13 with an inlet opening 11 and an outlet opening 12, which defines the receiving volume P. The inlet opening 11 can be in or be brought into fluidic contact with the surroundings of the device or the gas measuring device 200, or can be in or be brought into fluidic contact with a gas supply device (not shown), such as a mouthpiece of the gas measuring device 200. The outlet opening 12 can be in direct or indirect fluidic connection with the intake element 20 or can be brought into said connection by optionally opening an optional closing element, e.g. a valve. The sampling element 10 can, for example, be configured to receive the gas sensor 60 of the gas measuring device 200. The gas sensor 60 can, for example, be designed as an electrochemical gas sensor 60 or as an infrared optical gas sensor 60, although this is not necessary. It is also possible to provide a plurality of gas sensors as part of the gas measuring device 200. For example, for this purpose the sampling element 10 can receive an electrochemical gas sensor and an infrared optical gas sensor or be in fluidic connection with them.

The device 100 or the gas measuring device 200 can include a control and evaluation unit 50, which can be configured to control the operation of the device 100 and/or the gas measuring device 200 and optionally to perform data evaluation of the measurement data that can be obtained using the gas measuring device 200. In particular, the control and evaluation unit 50 can be configured to receive measurement data from the gas sensor(s) 60 and/or to control the actuator 30. The gas measuring device 200 may also include other known components such as an electrical energy storage device (e.g. accumulator, battery), a data storage unit, one or more data interfaces, and/or a display, and the like.

The control and evaluation unit 50 can, for example, include one or more processor(s), microprocessor(s), multicore processor(s), coprocessor(s), digital signal processor hardware (DSP hardware), network processor(s), application-specific integrated circuit(s) (ASIC), field-programmable gate array(s) (FPGA), read-only memory (ROM) for storing software, random access memory (RAM), and/or non-volatile storage device(s). The control and evaluation unit 50 can include other hardware-conventional and/or customer-specific.

The intake element 20 can, for example, be designed as a bellows, as shown in FIG. 1a, 1b.

The actuator 30 can have a shaft 41 and be configured to drive the shaft 41 linearly or approximately linearly. For this purpose, the actuator 30 can, for example, be designed as a lifting magnet 30, which can provide a drive movement of the shaft 41 in the positive x-direction.

In the exemplary embodiment according to FIG. 1a, 1b, the mechanism 40 comprises the shaft 41, which is linearly movable relative to the frame 44 of the device 100 or the gas measuring device 200 and which is rigidly connected to the intake element 20, a tensioning element 42 having an engagement projection 46 and being connected to the shaft 41 and thus being movable relative to the frame 44, a spring 45 and a locking element 43, which in the exemplary embodiment according to FIG. 1a, 1b is pivotably mounted in the frame 44. Thus, in the exemplary embodiment according to FIG. 1a, 1b the mechanism 40 is designed as a tensioning-and-latching mechanism 40, namely as a releasable tensioning-and-latching mechanism 40. In this case, the device 100 is configured such that the spring 45 stores mechanical energy by means of a first actuation of the mechanism 40 by the actuator 30 from a rest position (state according to FIG. 1b, spring travel 11) to a locking position (state according to FIG. 1a, spring travel 12) and, by means of blocking the tensioning element 42 by the locking element 43, locking of the movement of the shaft 41, i.e. locking of the tensioning element 42 in the locking position, is brought about. The spring 45 can thus attempt to return the shaft 41 counter to the drive movement of the actuator 30, i.e. in the negative x-direction in FIG. 1a, 1b. By releasing the blocking, i.e. by disengaging the tensioning element 42 and locking element 43, for example by actuating the locking element 43, the stored mechanical energy is released in the return direction F of the spring 45 as kinetic energy and thus causes the shaft 41 to return to its rest position. The actuation of the locking element 43 can be triggered, for example, by the control and evaluation unit 50. The locking element 43 can, for example, be actuated by an additional actuator (not shown) in order to cause a pivoting out of the blocking engagement of tensioning element 42 and locking element 43, cf. in FIG. 1a the direction of movement of the locking element 43 schematically indicated by an arrow.

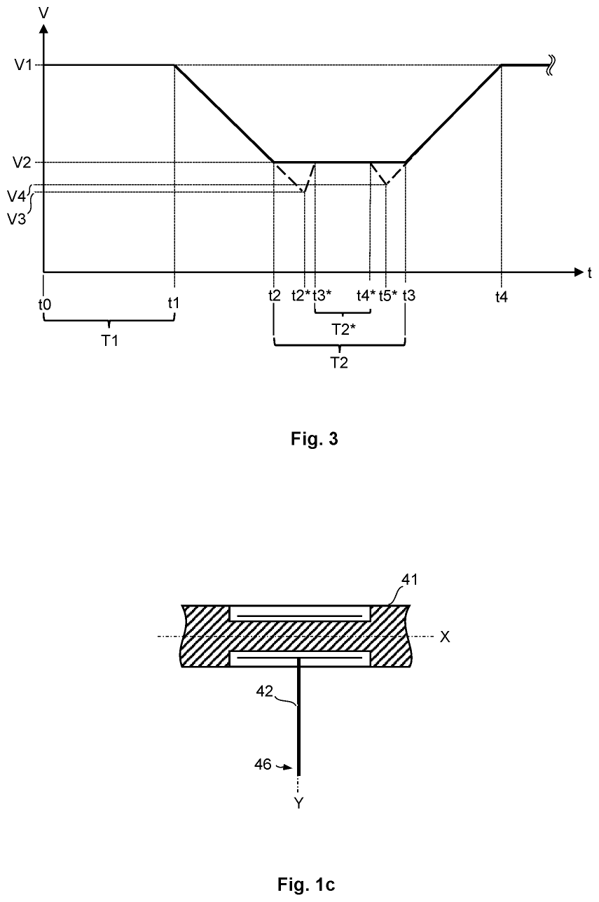

FIG. 3 illustrates, as an intake volume V/time t graph, how sampling can be carried out with the device 100 or the gas measuring device 200 according to the present disclosure. In this case, the solid line represents the sequence of the exemplary embodiment according to FIG. 1a, 1b. As described above, in a first position, namely in the rest position (FIG. 1b), the mechanism 40 provides the first volume V1 as the intake volume V. This can be done for a predetermined first period of time T1, which can extend from a first time t0 to a second time t1. During this period of time T1, actuator 30 may be deactivated. At time t1, the actuator 30 can be activated by the control and evaluation unit 50 and drive the shaft 41 along the longitudinal axis X, i.e. in the positive x-direction, as described above. In this case, the intake volume V is reduced starting from the first volume V1 until it reaches the second volume V2 in the second position, namely in the locking position (FIG. 1a). This volume can be reached at time t2. The actuator 30 can now be deactivated for the second period of time T2, i.e. from time t2 to time t3, since the mechanism 40 is locked as described above. After the second period of time T2 has elapsed, the locking can be released, so that the mechanism 40 returns to its rest position as described above, i.e. the intake volume V can, proceeding from the second volume V2, assume the first volume V1 again, which may have occurred at time t4. The first volume change V1 to V2 can, for example, be used to displace the gas G located in the receiving volume P, in order to prepare the device 100 for a measurement. The second volume change V2 to V1 can be used, for example, to effect a suctioning sampling, through which the gas G to be measured enters the receiving volume P of the sampling element 10. Thus, the device 100 enables measuring operation of the gas measuring device 200.

The tensioning element 42 can be rigidly connected to the shaft 41, as shown in the exemplary embodiment according to FIG. 1a, 1b. Alternatively, in one exemplary embodiment of the device 100, it is possible that the tensioning element 42 is movably mounted on the shaft 41. An example of such a mounting is shown in FIG. 1c. In this case, the tensioning element 42 with the engagement projection 46 is pivotably received on the shaft 41. The possible pivot range can be limited by a stop on one side or two sides. The alternative exemplary embodiment can be combined with the exemplary embodiment according to FIG. 1a, 1b, it being possible for the rigid connection of tensioning element 42 and shaft 41 to be replaced by the described pivotable mounting, for the combination.

The locking element 43 can be pivotably received in the frame 44, as shown in the exemplary embodiment according to FIG. 1a, 1b. Alternatively, in one exemplary embodiment of the device 100, it is possible that the locking element 43 is designed to be fixed in position relative to the frame 44. One such exemplary embodiment is shown in FIG. 2a, 2b, according to which the locking element 43 is designed as a groove cam 43 in the frame 44. This alternative exemplary embodiment can be combined with the exemplary embodiment according to FIG. 1a, 1b by replacing the movable locking element 43 according to FIG. 1a, 1b with the groove cam 43 according to FIG. 2a, 2b. In this exemplary embodiment, the tensioning element 42 is therefore engaged with the groove cam 43 by means of the engagement projection 46, as can be seen in FIG. 2a, 2b. In this case, the groove cam 43 is designed such that the tensioning element 42 or its engagement projection 46 is in engagement with the groove cam 43 at every point of the groove cam 43, by the groove cam 43 running below a frame surface 44a at every point of the groove cam 43, so that the engagement projection 46 cannot slide out of the groove cam 43 when viewed in the y-direction.

The groove cam 43 has, as shown in FIG. 2a, 2b, at least a first region a for providing a rest position i of the tensioning element 42, and a second region b for providing a locking position ii of the tensioning element 42. The rest position i corresponds to a first volume V1 of the intake volume V, and the locking position ii corresponds to a second volume V2 of the intake volume V. Furthermore, the groove cam 43 can optionally have a third region c and a fourth region d, as shown in FIG. 2a. The third region c serves to provide an intermediate position iii of the locking mechanism 40, which corresponds to an optional third volume V3 of the intake volume V. The fourth region d serves to provide a further intermediate position iv of the locking mechanism 40, which corresponds to an optional fourth volume V4 of the intake volume V.

In the exemplary embodiment according to FIG. 2a, 2b, it is preferred that the tensioning element 42 is pivotably mounted on the shaft 41, so that the tensioning element 42 has, relative to the shaft 41, one degree of freedom transverse to the shaft 41, i.e. in the z-direction in FIG. 1a, 1b, 2a, 2b. For this purpose, the tensioning element 42 can be designed according to the exemplary embodiment shown in FIG. 1c. This allows the tensioning element 42 or engagement projection 46 to follow the contour of the groove cam 43.

In the exemplary embodiment shown in FIG. 2a, 2b, the groove cam 43 is preferably designed such that a movement of the tensioning element 42 is possible only in one direction, namely from the first region a to the third region c, from the third region c to the second region b, from the second region b to the fourth region d, and from the fourth region d to the first region a, but not in any other direction. For this purpose, the groove cam 43 can have a projection 47a, 47b, 47c, 47d in some or all regions a, b, c, d, which projection reduces one degree of freedom of the tensioning element 42 in the respective region a, b, c, d, in that the movement of the tensioning element 42 in an undesired direction is blocked by the projections 47a, 47b, 47c, 47d.

FIG. 3 illustrates, as an intake volume V/time t graph, how sampling can be carried out with the device 100 or the gas measuring device 200 according to the present disclosure. In this case, the dashed line represents the sequence of the exemplary embodiment according to FIG. 2a, 2b.

As described above, the mechanism 40 provides the first volume V1 as the intake volume V in a first position, namely in the rest position i. This can be provided for a predetermined first period of time T1, which can extend, for example, from a first time t0 to a second time t1. During this period of time T1, the actuator 30 can be deactivated, i.e. currentless for example. At time t1, the actuator 30 can be activated, e.g. by the control and evaluation unit 50, to drive the engagement projection 46 in the positive x-direction, so that the engagement projection 46 follows the contour of the groove cam 43 until reaching the third region c. An intermediate state of the engagement projection 46 in a position between the first region a and the second region b is shown in FIGS. 2a and 2b respectively. During this movement, the intake volume V is reduced starting from the first volume V1 until it reaches a third volume V3 in the third region c in the intermediate position iii. This can be the case at time t2*. The actuator 30 can now be deactivated, as a result of which the mechanical energy stored in the spring 45 during its deflection is partially released and converted into kinetic energy, thus driving the engagement projection 46 in the negative x-direction. In this case, due to the projection 47b of the groove cam 43 in the region c (intermediate position iii), only a movement in the direction of the second region b (rest position ii) along the contour of the groove cam 43 is possible. Driven by the spring 45, the engagement projection 46 thus follows the groove cam 43 as far as the second region b (rest position ii). There, the movement of the engagement projection 46 is interrupted despite the continuing spring force, since a form-fitting connection of the engagement projection 46 occurs in the second region b (rest position ii), which is designed as a depression with respect to the x-direction, against the restoring force of the spring 45 in the negative x-direction (stable or metastable position), and prevents further movement of the engagement projection 46. In the second region b, the intake volume V at time t3* has the second volume V2, as shown in FIG. 3. Thus, the engagement projection 46 initially remains in the second region b (rest position ii). This can be done, for example, for the predetermined second period T2*, i.e. from time t3* to a time t4*. If, for example, a further change in volume is desired after the predetermined second period T2* has elapsed, the actuator 30 can be actuated again to cause a drive movement of the engagement projection 46 in the positive x-direction. Since the second region b (rest position ii) has the projection 47c, the engagement projection 46 of the groove cam 43 can follow only in the direction of the fourth region d (further intermediate position iv). The engagement projection 46 can reach the fourth region d (further intermediate position iv) for example at time t5*, see FIG. 3. In this case, the intake volume V has the fourth volume V4. The actuator 30 can now be deactivated again, so that the mechanical energy stored in the spring 45 is converted into kinetic energy as a drive for the engagement projection 46, by restoring the spring 45 in the negative x-direction. Due to the projection 47d and the contour of the groove cam 43, the engagement projection 46 follows the groove cam 43 back into the first region a (rest position i), in which the intake volume V at time t4 again has the first volume V1 (see FIG. 3). Starting from this rest position i, the previously described movement sequence can optionally be performed again.

The change in volume of the intake volume V from the first volume V1 to the third volume V3 can be used in the gas measuring device 200, for example, to displace the gas G located in the intake volume P and to prepare the gas measuring device 200 for a gas measurement. The change in volume of the intake volume V from the third volume V3 to the second volume V2 can then be used in the gas measuring device 200 for example to carry out an initial sampling, for example to determine a mouth alcohol concentration. The change in volume of the intake volume V from the second volume V2 to the fourth volume V4 can be used in the gas measuring device 200 for example to (partially) displace, again, the gas G previously taken as a sample. The change in volume of the intake volume V from the fourth volume V4 to the first volume V1 can then be used for example to carry out a second sampling, for example to determine a blood alcohol concentration. Although FIG. 3 shows that the third volume V3 can be smaller than the fourth volume V4, this is not necessary. For example, the third volume V3 can also be larger than the fourth volume V4 or the same size.

As can be seen from the foregoing, in each exemplary embodiment of the present disclosure, the mechanism 40 has at least two stable or metastable positions, namely the rest position and the locking position. The mechanism 40 can be adapted in each exemplary embodiment so as to provide additional stable or metastable positions. In the exemplary embodiment shown in FIG. 1a, 1b, this can be achieved, for example, by providing additional locking elements 43 along the x-direction, which, at different positions of the x-axis, provide locking of the shaft 41 with corresponding different intake volumes V. In the exemplary embodiment according to FIG. 2a, 2b, this can be achieved by providing further regions designed as depressions in which the engagement projection 46 comes to rest, against the restoring force of the spring 45, without the provision of a driving force by the actuator 30.

In general, by providing the described mechanism 40, the deflection of the shaft 41 in the working cycle before reaching the next position, as well as the position of the shaft 41 in the respective position and thus the distances traveled between the respective positions in each working cycle, can be predetermined substantially arbitrarily via the design of the mechanism 40. Thus, various well-defined distances can be covered in succession, and well-defined changes in volume of the intake volume V can thus be provided, without the actuator 30 itself requiring position control. In this case, the next defined change in volume in each case can be triggered by activating actuator 30 without position control.

All features and embodiments disclosed herein may be combined with one another as desired, provided this does not relate to alternatives and is not contradictory.

List of reference signs

| | |
|---|---|
| 100 | device |
| 200 | gas measuring device |
| 10 | sampling element |
| 11 | inlet opening |
| 12 | outlet opening |
| 13 | housing |
| 20 | intake element that can change in volume, bellows |
| 30 | actuator |
| 40 | mechanism |
| 41 | shaft |
| 42 | tensioning element |
| 43 | locking mechanism, groove cam |
| 44 | frame |
| 44a | frame surface |
| 45 | spring element |
| 46 | engagement projection |
| 47a, 47b, 47c, 47d | projections |
| 50 | control and evaluation unit |
| 60 | gas sensor |
| a | first region |
| b | second region |
| c | third region |
| d | fourth region |
| F | spring force |
| G | gas |
| i | rest position |
| ii | locking position |
| iii | intermediate position |
| iv | further intermediate position |
| I1 | first spring travel |
| I2 | second spring travel |
| P | receiving volume |
| T1 | first period of time |
| T2 | second period of time |
| t0, t1, t2, . . . | times |
| V | intake volume |
| V1 | first volume |
| V2 | second volume |
| V3 | third volume |
| V4 | fourth volume |
| X | longitudinal axis |
| Y | axis of engagement of the tensioning element |
| x, y, z | spatial axes, directions |

1-10. (canceled)

11. A device for sampling a gas, the device comprising:
a sampling element that defines a receiving volume for receiving a sample of the gas;
an intake element that provides a variable intake volume and that is fluidically connected to the receiving volume; and
an actuator that is configured to change the intake volume via a locking mechanism and thereby to aspirate a sample into the receiving volume,
wherein the locking mechanism includes a locking transmission comprising a first transmission element configured to be temporarily locked relative to a second transmission element.

12. The device of claim 11, wherein:
a first volume of the intake volume is provided by the locking mechanism for a predetermined first period of time, and
wherein a second volume of the intake volume is provided by the locking mechanism for a predetermined second period of time.

13. The device of claim 12,
wherein the locking mechanism is designed as a tensioning-and-latching mechanism.

14. The device of claim 13,
wherein the tensioning-and-latching mechanism is designed as a releasable tensioning-and-latching mechanism.

15. The device of claim 13,
wherein the tensioning-and-latching mechanism comprises a tensioning element that is movable by the actuator and configured to be engaged with a locking element that is fixed or movable relative to a frame of the tensioning-and-latching mechanism.

16. The device of claim 15,
wherein the locking element is fixed relative to the frame and is designed as a groove cam.

17. The device of claim 16, wherein:
the tensioning element is mounted on a shaft of the actuator and comprises an engagement projection engaged with the groove cam,
wherein the groove cam comprises:
a first region for providing a rest position of the tensioning element, the first region corresponding to the first volume of the intake volume, and
a second region for providing a locking position of the locking mechanism, the second region corresponding to a second volume of the intake volume.

18. The device of claim 17, wherein the groove cam further comprises a third region for providing an intermediate position of the locking mechanism, the third region corresponding to a third volume of the intake volume.

19. The device of claim 18, wherein the groove cam further comprises a fourth region for providing a further intermediate position of the locking mechanism, the fourth region corresponding to a fourth volume of the intake volume.

20. The device of claim 12,
wherein a third volume of the intake volume is provided by means of the locking mechanism.

21. The device of claim 20, wherein a fourth volume of the intake volume is provided by the locking mechanism.

22. A gas measuring device for qualitative or quantitative determination of a component in a breathing gas sample by means of a gas sensor, the gas measuring device comprising a device for sampling a breathing gas, wherein the device for sampling the breathing gas comprises:
a sampling element that defines a receiving volume for receiving a sample of the gas;
an intake element that provides a variable intake volume and that is fluidically connected to the receiving volume; and
an actuator that is configured to change the intake volume via a locking mechanism and thereby to aspirate a sample into the receiving volume,
wherein the locking mechanism includes a locking transmission comprising a first transmission element configured to be temporarily locked relative to a second transmission element.

23. The gas measuring device of claim 22,
wherein the sampling element is configured to:
receive the gas sensor, or
provide a fluidic connection between the receiving volume and the gas sensor.

24. The gas measuring device of claim 22, wherein:
a first volume of the intake volume is provided by the locking mechanism for a predetermined first period of time, and
wherein a second volume of the intake volume is provided by the locking mechanism for a predetermined second period of time.

25. The gas measuring device of claim 24,
wherein the locking mechanism is designed as a tensioning-and-latching mechanism.

26. The gas measuring device of claim 25,
wherein the tensioning-and-latching mechanism is designed as a releasable tensioning-and-latching mechanism.

27. The gas measuring device of claim 25,
wherein the tensioning-and-latching mechanism comprises a tensioning element that is movable by the actuator and configured to be engaged with a locking element that is fixed or movable relative to a frame of the tensioning-and-latching mechanism.

28. The gas measuring device of claim 27,
wherein the locking element is fixed relative to the frame and is designed as a groove cam.

29. The gas measuring device of claim 28, wherein:
the tensioning element is mounted on a shaft of the actuator and comprises an engagement projection engaged with the groove cam,
wherein the groove cam comprises:
a first region for providing a rest position of the tensioning element, the first region corresponding to the first volume of the intake volume, and
a second region for providing a locking position of the locking mechanism, the second region corresponding to a second volume of the intake volume.

30. The gas measuring device of claim 24, wherein a third volume of the intake volume is provided by means of the locking mechanism.
